(19) United States
(12) Patent Application Publication
(10) Pub. No.: US 2024/0385178 A1
(43) Pub. Date: Nov. 21, 2024

(54) TRAINED NEURONAL CELL CULTURES FOR DRUG TESTING

(71) Applicant: MICROSOFT TECHNOLOGY LICENSING, LLC, Redmond, WA (US)

(72) Inventors: Weiwei YANG, Seattle, WA (US); Benjamin Franklin CUTLER, Seattle, WA (US); Christopher Miles WHITE, Redmond, WA (US); Whitney HUDSON, Woodinville, WA (US)

(21) Appl. No.: 18/236,366

(22) Filed: Aug. 21, 2023

Related U.S. Application Data
(60) Provisional application No. 63/503,400, filed on May 19, 2023.
(60) Provisional application No. 63/503,655, filed on May 22, 2023.
(60) Provisional application No. 63/503,406, filed on May 19, 2023.

(51) Int. Cl. G01N 33/50; C12M 1/22; C12M 1/34; C12N 5/0793

(52) U.S. Cl. CPC G01N 33/5073; C12M 23/10; C12M 41/46; C12N 5/0619

(57) ABSTRACT

This disclosure provides techniques and systems for using neuronal cell cultures such as organoids as a testing platform for drug development. The neuronal cell cultures are trained to perform goal-oriented behavioral tasks. The ability of the neurons to perform a task is identified through connections to a computing device that provides inputs and measures outputs from the neurons. The neuronal cell culture is then exposed to a potential drug while evaluating its ability to perform the task. The performance of the neuronal cell culture in the presence and absence of the drug is compared and this provides the basis for evaluating the effects of the drug. Better performance on the task may indicate efficacy of the drug while decreased competency decreased competency may indicate toxicity. Personalized testing can be provided by using induced pluripotent stem cells generated from a patient's own somatic cells.

### PRIORITY APPLICATIONS

This application claims the benefit of and priority to U.S. Provisional Application No. 63/503,400, filed May 19, 2023; U.S. Provisional Application No. 63/503,406, filed May 19, 2023; and U.S. Provisional Application No. 63/503,655, filed May 22, 2023, the entire contents of which are incorporated herein by reference.

### BACKGROUND

Currently, drug development involves testing new potential drugs using one or all of three methods: in vitro, in vivo, and in silico testing. In vitro testing uses cell cultures to test the effects of a potential drug and relies on established cellular physiology indicators derived from the functions and processes that occur within individual cells. However, these cellular physiology indicators are usually too low-level to provide a comprehensive evaluation of the efficacy and dosage of a treatment or drug.

Organoids, such as human cortical organoids (HCOs), are one type of cell culture that can be used for in vitro testing. However, even with specialized cell cultures such as HCOs, studies are limited to measuring action potentials recorded using sensors such as multi-electrode arrays (MEA) and comparing perturbed states with baseline states in terms of spike density calculated from counts of action potentials. This does not fully leverage the three-dimensional (3D) structure of organoids to understand drug and treatment effects on a functional level.

In vivo testing, which involves trials or testing that is performed on a living organism, such as a human or an animal, can provide more comprehensive results for assessing the safety and efficacy of drugs than in vitro testing. However, there are significant ethical and resource constraints associated with in vivo testing. Conducting in vivo experiments on humans raises ethical concerns regarding informed consent, privacy, and potential harm. Additionally, in vivo testing tends to be more expensive and time-consuming than in vitro testing, making it difficult to scale up and deploy as broadly as would be necessary for personalized medicine.

In silico modeling and simulation use computer models to simulate the behavior of biological systems, enabling researchers to evaluate the safety and efficacy of drugs and treatments without the need for expensive and time-consuming in vivo or in vitro experiments. However, building accurate and reliable models to capture complex, non-linear biological systems and processes is not a trivial task. The usefulness and applicability of in silico modeling greatly depend on the accuracy of the model. Moreover, there are ethical and regulatory challenges associated with in silico modeling, such as concerns about the use of patient data for building models, as well as the potential for bias in the models.

Improved or additional techniques for testing potential drugs are needed. This disclosure is made with respect to these and other considerations.

### SUMMARY

This disclosure provides drug testing systems and techniques to test drugs on neuronal cell cultures trained to perform goal-oriented behavioral tasks. These tests evaluate how a drug affects the ability of neuronal cell culture to either perform a learned task or to learn a new task. Evaluating how a drug affects the competency or learning of a neuronal cell culture can reveal more subtle effects of the drug than simply observing changes in spiking patterns of the neuron. Thus, the system and techniques of this disclosure provide a superior and novel way to test the effects of potential drugs.

A neuronal cell culture is trained to perform a goal-oriented behavioral task by connecting the cell culture to a computing device, providing stimulus to the neuronal cell culture, and detecting the response. The stimulus is used to provide feedback that shapes the behavior of the neurons in the neuronal cell culture. Without being bound by theory, it is believed that during training neural pathways are formed and strengthened between the neurons in neuronal cell cultures. Electrical signals generated by the neurons, as detected by the sensors, are interpreted by the computing device as signals used to perform the goal-oriented behavioral task. Thus, the neuronal cell culture performs a “task” by generating firing patterns that are detected and interpreted by the computing device.

In one implementation, a neuronal cell culture is grown on top of a MEA. The MEA can function both as a sensor for detecting electrical signals generated by the neurons and as a source of stimulus by providing small electrical shocks. However, other types of sensors can also be used. Stimulus can also be provided by applying a light, sound, motion, heat, or chemical, other than the drug being tested, to the neurons. At the start of training, the neuronal cell culture may generate action potentials relatively randomly and be unable to complete a goal-oriented behavioral task. During training positive and/or negative feedback can be provided based on the neuronal cell culture's performance on the task. During training the neuronal cell culture may be able to learn which firing patterns result in positive feedback and which result in negative feedback. This may shape the behavior of the neuronal cell culture so that it effectively learns how to perform the goal-oriented behavioral task. Because the neuronal cell culture interacts with the outside world through signals detected by sensors that are connected to a computing device, in some implementations the goal-oriented behavioral task is a task performed in a computer simulation of a virtual environment. For example, the virtual environment could be a computer game or a machine learning control problem.

There are multiple ways that neuronal cell cultures can be used to test the effects of a potential drug. A single neuronal cell culture can be trained in the absence of the drug to establish a baseline ability for the goal-oriented behavioral task. Then, the drug can be applied to the neuronal cell culture and any changes in its ability to perform the task observed. An increase in the ability of the neuronal cell culture to perform the task (such as playing a game more successfully) could be interpreted as an indication that the drug has a positive effect. Conversely, if the ability of the neural cell culture to perform the goal-oriented behavioral task decreases, that may be interpreted as the drug being harmful or potentially toxic.

In a variation of the technique, multiple neuronal cell cultures created from the same cell line can be used for side-by-side comparisons. For example, two cell cultures could be created and trained in identical matters and then one exposed to the drug while one is not. Any difference in the ability of these two cell cultures to perform the goal-oriented behavioral task can be attributed to the presence of the drug.

In addition to testing how a drug affects the competency of the neuronal cell culture to perform a learned task, the effects of the drug on the ability to learn the task can also be tested. For example, two identical cell cultures can be created and both trained to perform the same goal-oriented behavioral task-one trained in the presence of the drug on the other trained without the drug. Differences in the resources needed for training (e.g., time) as well as competency at the task once trained can be compared. If the neuronal cell culture trained in the presence of the drug is able to learn the task faster, it can be inferred that the drug is beneficial. Conversely, if the neuronal cell culture trained with the drug is not able to perform the task even after completion of training with the same competency as the neuronal cell culture trained without the drug, it can be inferred that the drug is potentially harmful or toxic.

Multiple identical neuronal cell cultures can be created and used to perform a variety of different comparisons. For example, the drug can be applied at different concentrations in order to study the effect of dosage. Also, multiple neuronal cell cultures from the same cell lines can be trained to perform different goal-oriented behavioral tasks. There can be a pair of neuronal cell cultures created for each task-one with the drug and one without. These pairs of neuronal cell cultures each trained for a different task can effectively create a “battery of tests” to determine if any positive or negative effects of a drug are specific to a particular task or are broadly generalizable.

The methods and techniques of this disclosure have advantages compared to currently established methods of drug testing because they combine the ease of in vitro testing with higher behavioral functions similar to in vivo testing and offer the programmatic flexibility of in silico testing. In some instances, somatic cells collected from a patient may be reprogrammed to create induced pluripotent stem cells (iPSCs). This results in a neuronal cell culture that is personalized to the individual patient. This provides a basis for personalized medicine because the results of the testing will be specific to the neurons from that individual.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 shows a system for using a neuronal cell culture to test the effects of a drug.

FIG. 2 shows training of a real cell culture to perform a goal-oriented behavioral task in a virtual environment.

FIG. 3 shows comparative testing of two neuronal cell cultures, one in the presence of a drug and one without.

FIG. 4 shows an illustrative method for evaluating the effects of a drug on the competency of a trained neuronal cell culture to perform a goal-oriented behavioral task.

FIG. 5 shows an illustrative method for comparing the learning abilities of two neuronal cell cultures, one trained with the drug and one without.

FIG. 6 shows a computer architecture diagram illustrating a computing device architecture for a computing device capable of implementing aspects of the techniques and technologies presented herein.

### DETAILED DESCRIPTION

The techniques of this disclosure are based on using neuronal cell cultures, such as but not limited to organoids, as a platform for testing potential drugs. The neuronal cell cultures are trained to perform a goal-oriented behavioral task. This drug testing system evaluates how exposure to a drug affects a cell culture's ability to perform and/or learn the task. This type of testing can be used to compare different dosages of a drug, the effects on cells from different patients, as well as different drugs or combinations of drugs.

This is a more sophisticated drug testing technique than conventional in vitro testing which simply exposes a cell culture to a drug and observes a physical change. Conventional in vitro testing techniques often consider only superficial changes such as death of cells or a change in the firing rate of neurons. A test that simply counts the number or frequency of electrical spikes generated by action potentials may fail to distinguish between activity that indicates a stress response and a healthy firing pattern. Thus, currently in vitro techniques (even those using organoids) are limited to unsophisticated metrics for identifying the effect a potential drug has on neurons.

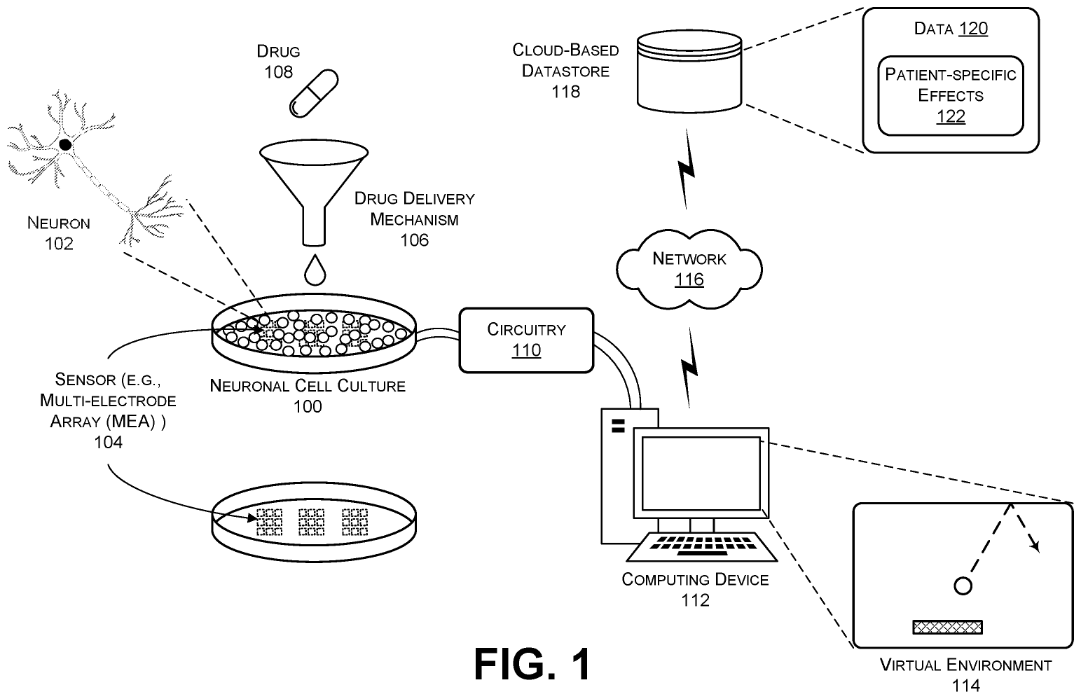

FIG. 1 shows a system that includes the neuronal cell culture 100 that can be trained to perform a goal-oriented behavioral task. There are many techniques known to those of ordinary skill in the art for culturing neurons 102 to grow outside of a living organism. The neurons 102 may be grown from embryonic stem cells or induced pluripotent stem cells (iPSCs). Induced pluripotent stem cells (iPSCs) are a type of pluripotent stem cell that can be generated directly from a somatic cell. They are derived from skin or blood cells that have been reprogrammed back into an embryonic-like pluripotent state that enables the development of an unlimited source of any type of human cell needed. The neurons 102 may be human or from a non-human animal such as a primate or rodent. For example, the neurons 102 may be human induced pluripotent stem cells (hiPSCs). As a further example, the neurons 102 may be harvested from embryonic rodent brains. A neuronal cell culture 100 will typically contain a large number of individual neurons 102 that all come from the same source and are genetically identical. Examples of suitable neurons 102 and techniques for creating neuronal cell cultures 100 are described in Cleber A. Trujillo et al., Complex Oscillatory Waves Emerging from Cortical Organoids Model Early Human Brain Network Development, 25 Stem Cell Reports 558 (2019).

The neuronal cell culture 100 may be a two-dimensional (2D) cell culture or a three-dimensional (3D) cell culture. In 2D cell cultures, cells are grown in a single layer on top of a flat surface, whereas in 3D cell cultures, the neurons 102 are grown in a 3D space. The formation of a cell monolayer in a 2D cell culture is faster and has lower reagent costs than in a 3D cell culture. However, 3D cell cultures mimic in vivo environments more closely. An organoid is a specific type of 3D cell culture. Cancer tumorspheres are another type of 3D cell culture.

An organoid is a miniaturized and simplified version of an organ produced in vitro in 3D that mimics the key functional, structural, and biological complexity of that organ. They are derived from one or a few cells from a tissue, embryonic stem cells, or induced pluripotent stem cells, which can self-organize in three-dimensional culture owing to their intrinsic properties and/or extrinsic cues provided by the culture environment. Organoids are 3D cell cultures that contain organ-specific cell types which exhibit spatial organization and replicate some functions of the organ. Cortical organoids may exhibit cortical folds.

Cortical organoids or brain organoids are one example of an organoid. Cortical organoids are derived from embryonic stem cells (ESCs) or iPSCs. Cortical organoids are created by culturing stem cells in a 3D rotational bioreactor and develop over months with cell types and cytoarchitectures that resemble an embryonic brain. The growth of a cortical organoid tends to reproduce the developmental path of the brain of a developing embryo. A cortical organoid grown from human cells is referred to as a human cortical organoid (HCO). Techniques for growing HCOs are known to those of ordinary skill in the art and described in Trujillo et al.

The neuronal cell culture 100 may be grown from human induced pluripotent stem cells (hiPSCs) of a specific patient creating a personalized cell culture. The hiPSCs of a patient may be used to create a 2D or 3D neuronal cell culture 100 such as an HCO. This creates a platform for personalized, patient-specific drug testing. It can be used to learn how that individual will likely respond to a drug without the risks of consuming the drug. It can also be used to identify a personalized dosage regimen.

The neuronal cell culture 100 is connected to one or more sensors 104 that are capable of detecting activities of the neurons 102. In one implementation, the sensors are one or more electrodes. For example, the sensors 104 may be implemented as an MEA. However, other types of sensors 104 and other electronic configurations are also possible. For example, thermal sensors may be used to detect changes in the temperature of the neurons 102 and optical sensors may be used to measure neural activity based on surface plasmon resonance. See Mitra Abedini et al., Recording Neural Activity Based on Surface Plasmon Resonance by Optical Fibers—A Computational Analysis, 12 Front. Comput. Neurosci., 16 Oct. (2018). When neurons 102 in the neuronal cell culture 100 generates an action potential, they produce electrical signals that can be detected by electrodes in the MEA 104. Examples of suitable MEAs, and techniques for growing a neuronal cell culture 100 on an MEA are known to those of ordinary skill in the art and described in Trujillo et al., Francesca Puppo et al., Super-Selective Reconstruction of Causal and Direct Connectivity With Application to in vitro iPSC Neuronal Networks, 15 Frontiers in Neuroscience (2021); and Brett J. Kagan et al., In vitro neurons learn and exhibit sentience when embodied in a simulated game-world, 23 Neuron 110, 3952-3969.e8 (2022), Kagan et al., and Tianyi Chen et al., Discovering a Change Point and Piecewise Linear Structure in a Time Series of Organoid Networks via the Iso-Mirror, Applied Network Science 8:45 (2023). An MEA, such as a high-density MEA, may have a large number of electrodes in contact with the neurons 102 of the neuronal cell culture 100 that can detect connectivity changes and action potential pattern changes with respect to each other. The ability to detect action potentials from individual or small groups of neurons 102 can be used to perform network analysis and identify changes in action potential patterns that could be indications of learning.

The electrodes in the MEA 104, or other electrodes in contact with the neuronal cell culture 100, may also provide stimulus to the neurons 102 in the form of electrical shocks. Use of stimulus or feedback to train the neuronal cell culture 100 is discussed in greater detail below. Thus, the MEA 104 can function as both a sensor to detect output from the neuronal cell culture 100 as well as a stimulator to provide input to the neuronal cell culture 100. Other stimulators or sources of stimulation may be used besides electrodes. For example, light, chemicals, sound, motion, and/or heat may be used to stimulate the neurons 102 in the neuronal cell culture 100. Light may be provided by LEDs, a projector, a digital micromirror, or other light source. In some implementations, the light may be provided as strobing light, for example light that strobes at a frequency of 10 Hz. Sound may be provided by one or more speakers. Motion may be provided by a vibrating tray, an agitator, or even by mechanically applying force (e.g., “poking”) neurons 102 in the neuronal cell culture 100.

The system for drug testing includes a drug delivery mechanism 106 that is configured to deliver a drug 108 to the neuronal cell culture 100. The drug delivery mechanism 106 may be any type of apparatus or device that is able to deliver a controlled amount of liquid or powder to cells in cell culture. For example, the drug delivery mechanism 106 may be implemented as a dropper or pipette including one that is operated manually by a human user. The drug delivery mechanism 106 may also be an automated or mechanized device such as laboratory robotics or microfluidics. The drug delivery mechanism 106 is able to control the volume and timing of drug delivery to the neuronal cell culture 100.

The drug 108 is any type of drug or pharmaceutical compound intended for testing on the neuronal cell culture 100. The drug 108 may be a drug that has been previously used on humans are other animals or it may be an untested drug that does not have regulatory approval. The drug 108 may be supplied in any form and processed, such as by dissolving in appropriate media, or application to the neuronal cell culture 100.

Circuitry 110 connected to the MEA 104 provides a connection between the electrodes of the MEA 104 and a computing device 112. The circuitry 110 may be any type of circuitry conventionally used to control an MEA 104 or alternative type of sensor and stimulator. Control systems for MEAs are known to those of ordinary skill in the art and described in Trujillo et al., Puppo et al., Kagan et al., and Chen et al. If a sensor other than an MEA 104 is used, the circuitry 110 is configured to connect that sensor to the computing device 112.

The computing device 112 can be any type of conventional computing device such as a desktop computer, laptop computer, tablet computer, smart phone, or the like. The computing device 112 may also be physically located at a distance from the neuronal cell culture 100. For example, the computing device 112 may be a network-accessible or cloud-based computing device with physical components spread across multiple different locations. In an implementation, the circuitry 110 is connected to the network 116 and signals from the circuitry 110 are conveyed through the network 116 to the computing device 112. The computing device 112 may include software and interpret signals received from the circuitry 110 so that it can compare spiking activity of neurons 102 in the neuronal cell culture 100 in the presence and absence of the drug 108. The computing device 112 may then be configured to determine how the performance of the neuronal cell culture 100 on the goal-oriented behavioral task changes in response to the drug 108.

In this illustration, only a single neuronal cell culture 100, circuitry 110, and computing device 112 are shown. However, this system for drug testing may include multiple instances of each such as a first and second neuronal cell culture 100, first and second circuitry 110, and first and second computing device 112. For example, there may be many tens, hundreds, or more neuronal cell cultures 100 each connected to the appropriate circuitry 110 so that signals can be sent and received from the computing device 112. The computing device 112 may also be implemented as multiple separate computing devices such as one computing device for every set number of neuronal cell cultures 100 (e.g., one computing device 112 for every 10 neuronal cell cultures 100).

The computing device 112 interprets signals from the MEA 104 provided via the circuitry 110 to determine if the neuronal cell culture 100 is able to perform a goal-based behavioral task. The neuronal cell culture 100 does not necessarily perform a “task” in the conventional sense of interacting with elements outside the cell culture but performs the task by creating a firing pattern of the neurons 102 that is recognized by the computing device 112. Thus, the goal-oriented behavioral task may be a task that is performed in a computer simulation of a virtual environment 114. For example, the task could be playing a simple computer game such as Pong. The neuronal cell culture 100 would play the game Pong by activating certain neurons 102 that are detected by specific electrodes in the MEA 104 and interpreted by the computing device 112 as a signal to move a paddle to the left or the right. Electrical signals generated by the MEA 104 can provide the neuronal cell culture 100 with information about the state of the virtual environment 114 such as the position of the paddle and the movement of the ball. One example of specific techniques for training a neuronal cell culture 100 to play a simple computer game is provided by Kagan et al. Persons of ordinary skill in the art will understand how to adapt these techniques for other goal-oriented behavioral tasks besides the game Pong.

The computing device 112 may be communicatively connected to a network 116 such as the Internet or other communications network. The network 116 provides a communicative connection between the computing device 112 and other computing resources such as a cloud-based data store 118. During drug testing, device 112 can record the results of the behavior of the neuronal cell culture 100 and the presence or absence of the drug 108. This can be done for multiple drugs 108 and multiple neuronal cell cultures 100. Thus, the computing device 112 (either implemented as a single device or as a collection of discrete devices) collects data 120 on the learning and behavior of the neuronal cell cultures 100 in the presence and absence of multiple different drugs 108. This data 120 may be stored in some form of computer-readable media locally on the computing device 112 and/or remotely in the cloud-based data store 118. The data 120 may include, for example, an identifier of the drug 108 such as its name or chemical structure, and an identifier of the source of neurons 102 in the neuronal cell culture 100 such as the type of organism from which the cells were harvested or created, and a result of the drug testing. Thus, the data 120 may provide a record of past drug tests performed with the trained neuronal cell cultures 100.

The data 120 may also include records of tests performed on neurons from specific patients. This subset of the data 120 may store patient-specific effects 122 that indicate the efficacy of a drug for an individual patient or how dosage for that patient may vary from a standard dosage regimen. There may be a separate record of patient-specific effects 122 stored in the data 120 for each patient. The record of patient-specific effects 122 provides information for that individual patient or healthcare provider to make personalized choices about the use of tested drugs. This is a source of data to use for implementing personalized medicine for that patient. Records of the patient-specific effects 122 may include an identifier of the patient such as a name. In some implementations, records of patient-specific effects 122 may be anonymized so that personally identifiable information is removed and merged with other records for the drugs. Thus, data 120 may contain patient-specific effects 122 from multiple individual patients as well as the results of drug testing using neuronal cell cultures 100 from animals or unidentified human sources.

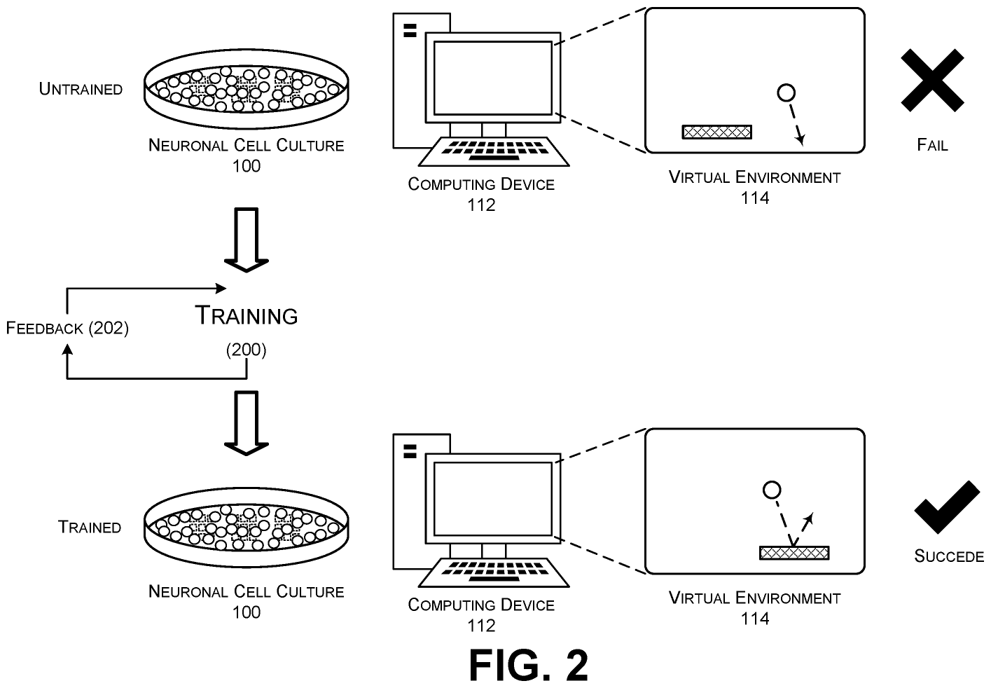

FIG. 2 shows an example of training a neuronal cell culture 100 to perform a goal-oriented behavioral task. An untrained neuronal cell culture 100 is created as described previously and connected to a computing device 112 that implements a virtual environment 114. The neuronal cell culture 100 is trained to perform a goal-oriented behavioral task so that the ability of the neuronal cell culture 100 perform the task or to learn the task can be tested with and without exposure to a potential drug.

The neuronal cell culture 100 comprises a large number of interconnected neurons that are communicating and transmitting signals to each other. Through training 200 the neurons collectively learn to perform a task that results in the formation and strengthening of neural pathways among the neurons. An untrained neuronal cell culture 100 will have spontaneous electrical activity and neurons in communication with neighboring cells. However, these untrained neurons will have low or no success with a goal-oriented behavioral task performed in a virtual environment 114.

The neuronal cell culture 100 is trained by exposure to outside stimuli that steers the cells in the neurons toward a desirable firing pattern. This firing pattern is then interpreted by the computing device 112 and used to affect a computer simulation in a virtual environment 114. A trained neuronal cell culture 100 is able to successfully perform the goal-oriented behavioral task. The goal-directed behavioral task is a predetermined task that the neuronal cell culture 100 is unable to perform before training.

This makes it possible to evaluate the effects of a drug against a functional metric namely the ability to perform (or learn) the goal-oriented behavioral task. Performing a functional task is more complicated than simply responding to a stimulus that results in firing activity of neurons. The trained neuronal cell culture 100 has a skill that can be performed with a variable level of success. This makes it possible to detect more subtle effects of a drug than conventional in vitro drug testing techniques. For example, the variable level of success of a neuronal cell culture on a task can be measured rather than simply the amount of neuron firing activity.

It has been previously established by Kagan et al. that neuronal cell cultures can be trained to learn to play simple games in a manner analogous to that of a neural network implemented in a conventional computing device. Kagan et al. demonstrated that approximately 800,000 brain cells grown in vitro could play a game similar to Pong using Friston's Free Energy Principle to design a reward signal for learning through supervised training. In neuroscience, the Free Energy Principle is based on the Bayesian idea of the brain as an “inference engine.” Under this principle, systems pursue paths of least surprise, or equivalently, minimize the difference between predictions based on their model of the world and their sense and associated perception. This difference is quantified by variational free energy and is minimized by continuous correction of the world model of the system, or by making the world more like the predictions of the system. Providing stimulation to the neurons that was “surprising” acted as a negative reward and providing structured stimulation that was “familiar” acted as a positive reward.

This demonstrates the ability of neuronal cell cultures to perform goal-directed behavioral tasks, highlighting the potential for neuronal cell cultures to learn. Once it is established that neuronal cell cultures can learn, it is then possible to adapt techniques from machine learning to teach classic control tasks that are inherently linked to innate motor and movement control. These classical machine learning tasks typically have a small and manageable set of states. Classic control tasks include CartPole in which the virtual environment 114 simulates a pole attached by an unactuated joint to a cart that moves along a frictionless track. The goal of the task is to balance the pole by applying forces in the left and right direction on the cart. A trained neuronal cell culture 100 may have groups of neurons that generate action potentials in specific regions corresponding to moving the cart to the left or to the right which is then interpreted by the computing device 112 as keeping the pole in balance. The ability or competency of the neuronal cell culture 100 with respect to the task can be measured by the length of time the cells can maintain the pole in an upright and vertical position. There are multiple other well-known control tasks used in conventional machine learning such as Arcobot and Pendulum.

The training 200 uses feedback 202 to shape the behavior of the neurons in the neuronal cell culture 100 so that a trained neuronal cell culture 100 can successfully perform the goal-directed behavioral task. However, a neuronal cell culture 100 may forget previous training with time and need to be retrained in order to maintain competency task. The feedback 202 can be provided in the form of electrical stimulation using or by application of chemicals to the neuronal cell culture 100. The feedback 202 may be positive or negative. One way of structuring positive or negative feedback is according to Friston's Free Energy Principle as described above. Other techniques for providing feedback 202 include, but are not limited to, providing light stimulus at a fixed frequency. The stimulus, whether it be through electricity, light, or another form, can be applied to one or more specific regions of a neuronal cell cultures 100.

The training 200 and feedback 202 can be implemented as a type of reinforcement learning (RL). Reinforcement learning is commonly used in computer science to train artificial intelligence systems to perform goal-oriented tasks. In RL, an agent is an entity that interacts with an environment by taking actions and receiving feedback in the form of rewards. The agent's goal is to learn an optimal policy for taking actions in the environment to achieve a target goal in the environment which can be represented by actions that maximize a cumulative reward over time. Updating the reward function informs the agent about the merits of a previous action in the context of the environment, and thus, enables the agent to select a next action.

In the context of RL, a state is a representation of the environment at a particular point in time. It provides information about the current situation that the agent can use to decide what action to take next. The state can include any information that is relevant to the agent's decision-making process, such as the current position of objects in the environment or the current score in a game. The state changes over time as the agent acts and interacts with the environment. When used for training neuronal cell cultures 100, the agent is the neuronal cell culture 100, the environment is the virtual environment 114 simulated by the computing device 112, and the state can be information about the virtual environment 114 (e.g., such as the position of a ball or the orientation of a pole). Information about the environmental states is sent from the computing device 112 to the neuronal cell culture 100 such as through activation of electric currents at specific electrodes in an MEA.

This type of training and the system for enabling the neuronal cell culture 100 to interact with the virtual environment 114 makes it possible to differentiate between a meaningful firing pattern and an arbitrary firing pattern that does not directly contribute to achieving a specified goal or task. A meaningful firing pattern is generally a firing pattern that results in successful performance on the goal-oriented behavioral task such as success in playing a game in the virtual environment 114. Without a defined and identified “goal,” it is not possible to determine if a firing pattern is meaningful. An arbitrary firing pattern would result in the neuronal cell culture 100 failing at the goal-oriented behavioral task. For example, a meaningful firing pattern and an arbitrary firing pattern that both result in the same frequency of action potentials would not be distinguished by conventional in vitro drug testing techniques. Conventional testing can detect changes in firing patterns, but has no metric to identify an effect of the changes without a goal.

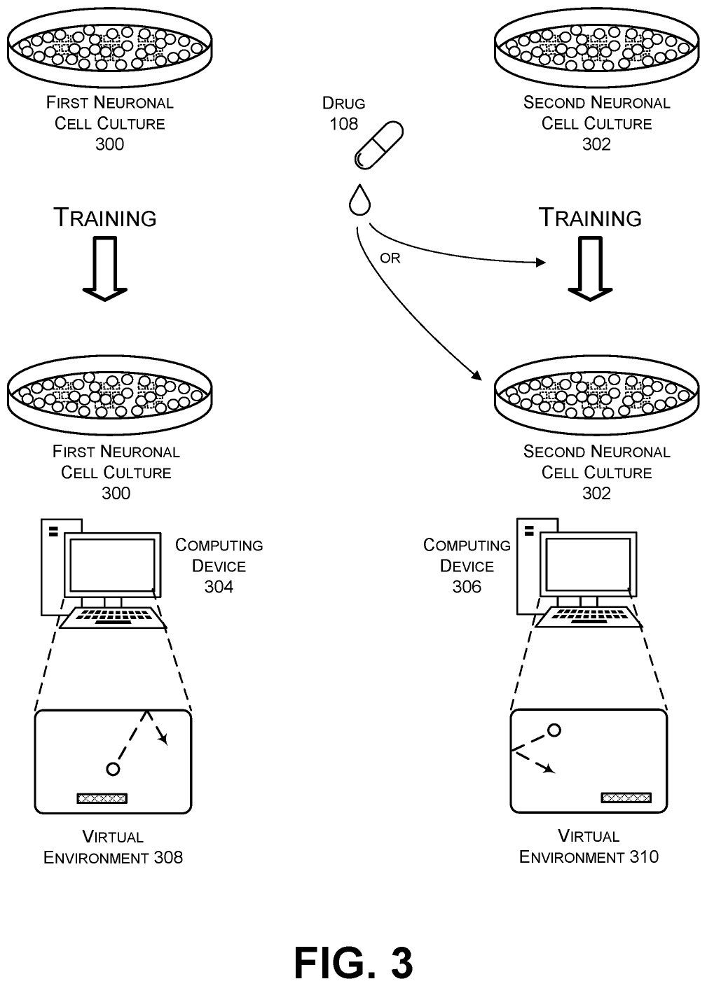

FIG. 3 shows comparative testing using a first neuronal cell culture 300 that is not exposed to the drug 108 and second neuronal cell culture 302 that is exposed to the drug 108. The first neuronal cell culture 300 and the second neuronal cell culture 302 are formed from cells of the same cell line so that they are genetically identical except possibly for epigenetic differences. Both the first neuronal cell culture 300 and the second neuronal cell culture 302 are trained to perform the same goal-oriented behavioral task as described previously. This creates two cultures of the same cells with the same training. Each may be connected to a respective computing device 304, 306 or they may be connected to the same computing device. In implementations in which the goal-oriented behavioral task is performed in a virtual environment, each of the neuronal cell cultures 300, 302 can perform the goal-oriented behavioral task in a separate virtual environment 308, 310. Thus, performance of each can be compared side-by-side with the only difference being exposure to the drug 108.

The first neuronal cell culture 300 functions as a control group and the second neuronal cell culture 302 is the exposed group. The drug 108 may be provided to the second neuronal cell culture 302 either during training or after training when attempting to perform the learned task. This provides for two possible types of comparisons. The first is a comparison of the effects of the drug 108 on the ability of the neuronal cell cultures 300, 302 to learn the goal-oriented behavioral task. The second is a comparison of the ability of trained neuronal cell cultures 300, 302 to perform a previously learned goal-oriented behavioral task. These two types of comparisons may be referred to as testing learning and testing competency.

A baseline for learning ability can be established from the first neuronal cell culture 300. The baseline may be the resources required to train the first neuronal cell culture 300 to perform the goal-oriented behavioral task with at least a threshold level of competency in the absence of the drug 108. This threshold could be, for example, achieving a certain score in the game of Pong or maintaining a pole in balance in CartPole for a set number of seconds. The resources can be any resource used to perform the training such as training time, number of training cycles, and amount of supervision. The training of the second neuronal cell culture 302 in the presence of the drug 108 can then be compared to the baseline. This testing can identify if the drug 108 improves, impedes, or has no effect on the ability of the second neuronal cell culture 302 to learn. The ultimate competency of the two cell cultures on the task can also be compared (i.e., first comparing learning ability and next comparing competency). Thus, how quickly the first neuronal cell culture 300 and the second neuronal cell culture 302 are able to learn the task as well as how well each can perform the task can be compared.

If both the first neuronal cell culture 300 and the second neuronal cell culture 302 are trained in the absence of the drug 108, the comparison is between the ability of the two cell cultures 300, 302 to perform the task with or without the drug 108. The performance of the first neuronal cell culture 300 on the task is the baseline. This testing can identify if the drug 108 improves, impedes, or has no effect on the ability of the second neuronal cell culture 302 to perform the task. The relative performance of the two cell cultures 300, 302 can be compared by a metric that is relevant for the particular goal-oriented behavioral task. For example, it could be points scored in a game or the length of time a pole is maintained in balance in CartPole.

A primary concern in drug testing may be identification of toxicity or detection of harm. These techniques are able to detect subtle indicators of harm such as decreased competency at a task or an increased time needed to learn a task. These types of subtle harms do not result in the neuronal cell culture being unable to perform the task or unable to learn the task but are indicative of a more subtle negative effect of the drug 108. This detects a type of harm that may be missed if the testing only considers death of cells or a decrease in firing activity. A harm or negative effect of a drug could be classified as a change relative to a baseline such as a 10% lower score on a game or 10% more training cycles needed to learn a task. The ability of cells in a neuronal culture to learn and perform tasks can be compared and rated on a scale. The ratings can be established based on a set of developed parameters.

Evaluating a drug to see if it provides a benefit can also be done in comparison to a baseline. If the second neuronal cell culture 302 becomes better at the task than the first neuronal cell culture 300 that may indicate an enhancement provided by the drug 108. Similarly, if the drug 108 is provided during training and the second neuronal cell culture 302 learns the task with fewer resources than the first neuronal cell culture 300 that may indicate a benefit. A benefit from the drug 108 may be classified as a change relative to a baseline (i.e., the performance of the first neuronal cell culture 300) such as a 10% higher score on a game or 10% fewer training cycles needed to learn a task.

Because the only significant difference for the purposes of drug testing between the first neuronal cell culture 300 and the second neuronal cell culture 302 is the presence of the drug 108, any difference in the ability of the two cell cultures to learn the task or competency in performing the task is attributable to the drug 108. This makes it possible to identify how the drug 108 affects the behavior of a neuronal cell culture and eliminate hidden variables that may be present in other types of drug testing. The ability to grow multiple neuronal cell cultures from the same cell line and provide the same training to each makes this testing technique reproducible and scalable.

Additionally, comparisons can be made between neuronal cell cultures 300, 302 based on skill transferability and the ability to learn a new task. For example, the first neuronal cell culture 300 and the second neuronal cell culture 302 can both be trained to perform a first goal-oriented behavioral task. They could be compared on this first task. Then starting with the trained neuronal cell cultures (in contrast to untrained neuronal cell cultures) both neuronal cell cultures 300, 302 would be trained to perform a second task. The comparison would evaluate how well the first neuronal cell culture 300 and the second neuronal cell culture 302 can learn to complete a new task after being trained to perform a first task. An example of this is testing the ability to solve the CartPole problem after successfully solving the Pendulum problem. This tests the effects of the drug 108 on the ability to retain previous neural connections formed by earlier training and to form new neural connections and change the learned task. Any of the evaluation metrics discussed above for learning a task or performing a task may be used.

Although only two neuronal cell cultures are shown in FIG. 3, comparative testing may be performed across a larger number of neuronal cell cultures. The behavior of the first neuronal cell culture 300 trained and evaluated in the absence of the drug 108 can serve as a baseline. The performance of the second neuronal cell culture 302, a third neuronal cell culture, a fourth neuronal cell culture, and potentially more can be compared to this baseline. This may be done to increase the sample size in order to increase accuracy. For example, the neuronal cell cultures evaluated in the presence of the drug 108 may use different amounts of the drug 108 to test the effects of dose.

Comparative testing across more than two different cell cultures can be used to create a battery of tests for evaluating the effects of the drug. For example, there could be multiple pairs of neuronal cell cultures, one exposed to the drug 108 and one not, each pair trained for a different goal-oriented behavioral task. Comparing the effect of the drug 108 on different tasks makes it possible to identify effects that are generalizable across many different tasks.

Similarly, multiple pairs of neuronal cell cultures could be created with cells from different individuals. As before, each pair of neuronal cell cultures would include one that is exposed to the drug 108 and one that is not. Each pair of neuronal cell cultures would be trained on the same goal-oriented behavioral task. Thus, the difference only between each pair of neuronal cell cultures is the source of the neurons. This type of training identifies effects of the drug 108 generalizable across different individuals and genetics.

A testing regimen could include many different batteries of tests with different baselines. For example, the drug 108 could be tested at different dosages, on a variety of different goal-oriented behavioral tasks, on the ability to learn the tasks and the competency to perform a learned task, and on cells from a variety of different sources. Other types of comparisons and tests are also possible. For example, different combinations of drugs could be compared. Multiple neuronal cell cultures can be prepared and trained with different combinations of drugs to identify drug interactions and sources of possible side effects. Baselines may be established based on the absence of all drugs and the presence of each drug alone. This provides a much more robust and complete picture of the effects of the drug 108 than any single test.

### Illustrative Methods

The illustrative methods of this disclosure may be implemented with the systems shown in any of FIGS. 1-3 or with alternative systems. Some steps in the methods may be optional and the order of the steps to be changed unless otherwise indicated by context.

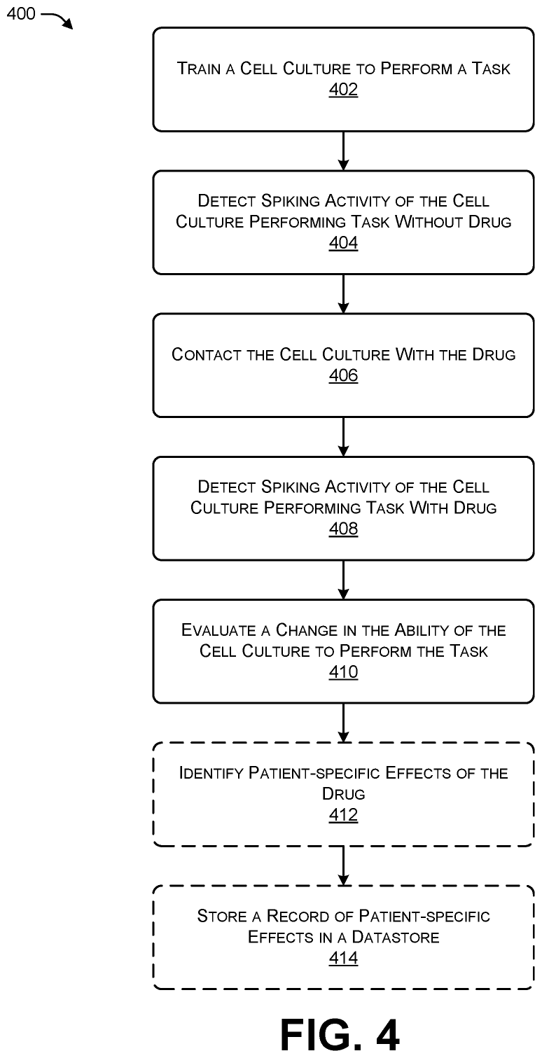

FIG. 4 shows a method 400 of using a neuronal cell culture trained to perform a goal-oriented behavioral task for testing the effects of a drug. Method 400 may be performed with a single neuronal cell culture by comparing behavior before and after contact with the drug.

At operation 402, the neuronal cell culture is trained to perform a goal-oriented behavioral task. The neuronal cell culture may be any type of neuronal cell culture discussed in this disclosure including, but not limited to, hiPSCs and non-human stem cells. The neuronal cell culture may be a 2D cell culture or a 3D cell culture. The goal-oriented behavioral task may be any type of behavioral task discussed in this disclosure such as a task performed in a computer simulation of a virtual environment.

The training may be any of the types of training discussed in this disclosure. For example, the training may be performed using reinforcement learning. Feedback for reinforcement learning may be provided by electrical stimulation. Electrical stimulation can be provided to neurons in the neuronal cell culture through electrodes such as electrodes in an MEA. In some implementations, feedback for reinforcement learning can be provided by the application of a chemical to the cell culture.

At operation 404, spiking activity of the neuronal cell culture is detected while the neuronal cell culture is performing the goal-oriented behavioral task in the absence of the drug. This establishes a baseline for the competency of the neuronal cell culture on the goal-oriented behavioral task. The spiking of the neurons in the cell culture can be detected by any suitable mechanism for detecting neuronal firing. For example, the spiking activity can be detected with electrodes such as electrodes of an MEA. The spiking activity as detected by the MEA can be passed through circuitry connected to a computing device where the computing device interprets the spiking activity. The computing device can then correlate the location and timing of the spiking activity with performance on the goal-oriented behavioral task.

At operation 406, the neuronal cell culture is contacted with the drug. The drug may be brought into contact with neuronal cell culture by any technique for applying a drug to a cell culture. For example, the drug may be placed into liquid media that is applied to or that surrounds the neuronal cell culture.

At operation 408, spiking activity of the neuronal cell cultures is detected while performing the goal-oriented behavioral task with the drug present. Detection of spiking activity may be performed as the drug is being applied, thus operations 406 and 408 can be performed simultaneously or substantially simultaneously. In another implementation, some amount of time is allowed to pass after operation 406 before the spiking activity is detected at operation 408. Thus, the neuronal cell culture is first contacted with the drug, there is time for the drug to affect the neurons, and the behavior of the neuronal cell culture is observed.

At operation 410, a change in the ability of the neuronal cell culture to perform the goal oriented behavioral task in response to the drug is evaluated. The change is evaluated by comparing the ability of the neuronal cell culture to perform the task before the drug with the ability of the neuronal cell culture to perform the task after exposure to the drug. Other conditions in the environment for the neuronal cell culture are maintained unchanged so that any difference can be attributed to the drug. An increase in the ability of the neuronal cell culture to perform the goal-oriented behavioral task may indicate that the drug is safe and effective. No change in the ability of the neuronal cell culture to perform the goal-oriented behavioral task may indicate the drug is safe but possibly does not provide any beneficial effect. A decrease in the ability of the neuronal cell culture to perform the goal-oriented behavioral task may indicate that the drug is harmful.

In some implementations, the cells of a specific patient, such as hiPSCs, may be cultured to create a neuronal cell culture that provides results personalized for the specific patient. In such implementations, the method 400 may include operation 412 during which patient-specific effects of the drug are identified. To create a personalized neuronal cell culture, somatic cells of a patient may be collected such as with a cheek swab. These somatic cells are then reprogrammed into induced pluripotent stem cells using known techniques. The induced pluripotent stem cells are directed to grow into neurons. Neuronal cell cultures, such as organoids, may be made from the neurons. These neuronal cell cultures will have the patient's genetic composition.

With patient-specific cells, the drug testing techniques of this disclosure can be used for personalized medicine. Using a personalized neuronal cell culture avoids the ethical risks of actually testing a drug on the patient. Yet it provides some information about how the drug may affect this particular patient differently from other people. This knowledge can be used to decide on appropriate treatment for the patient as a form of personalized medicine. For example, multiple different treatments (e.g., different dosages and/or different drugs) can be compared and see which one provides the most positive effect on a patient-specific neuronal cell culture.

Personalized neuronal cell cultures for multiple individuals can also be created and compared. These collections can be used to evaluate the effects of a drug across neurons with different genes. Techniques for making the cell cultures and for training may be kept constant so that the only variable is the source of the cells. This can provide information regarding how a drug will affect a cross-section of individuals without actually testing on a large group of people. Thus, cell cultures can be used for testing on either a personalized, non-personalized, or population basis.

At operation 414 a record of the patient-specific effects is stored in a datastore such as the cloud-based datastore 118 shown in FIG. 1. The datastore may contain records of patient-specific effects of multiple different patients. When used for purposes other than providing care to a specific patient, any personally identifiable information may be removed from the records. However, codes or unique identifiers may be used to identify individual patients so that the records in the datastore can be integrated into a real-world evidence (RWE) personalized medicine platform. Using a cloud-based datastore makes it easy to automate and share the records. Thus, these techniques may facilitate high throughput testing and improve the drug development process.

Baseline behaviors of neuronal cell cultures in the absence of a drug may also be stored in the datastore. Multiple different baselines may be established and stored. There may be individual baselines established for neuronal cell cultures from a particular individual. There may also be population baselines (e.g., such as average values or responses) for neuronal cell cultures derived from cells of multiple different individuals. Thus, the datastore may provide a basis for comparison when a drug is tested on a neuronal cell culture from a new patient.

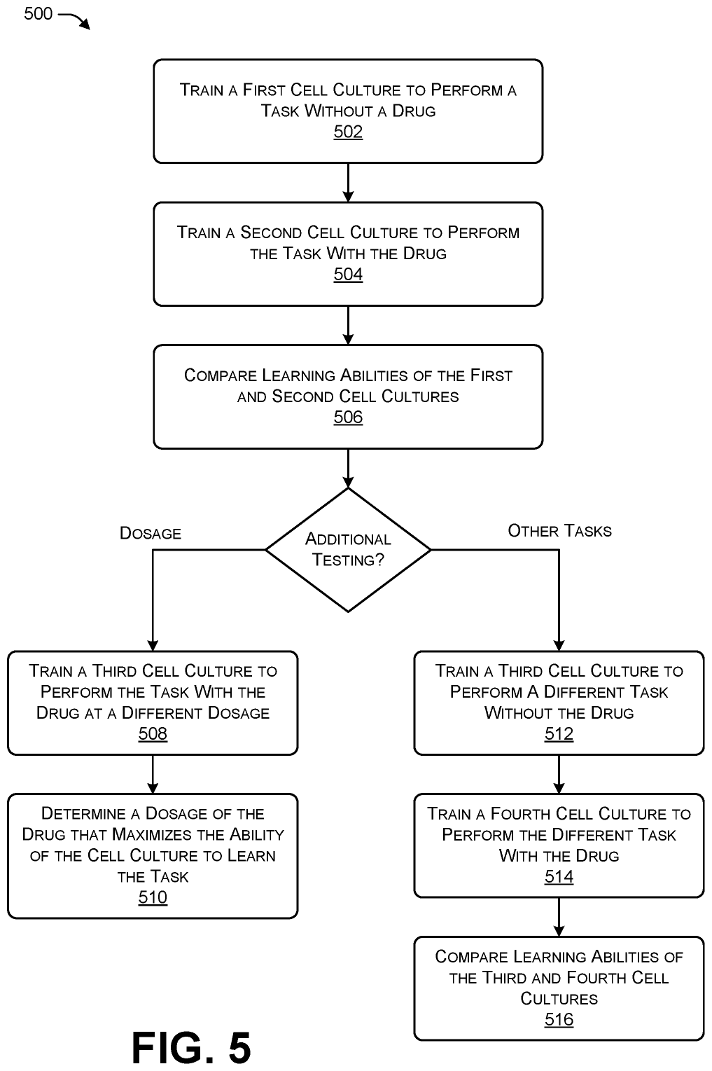

FIG. 5 shows a method 500 of comparing the abilities of a first and second neuronal cell culture to learn a goal-oriented behavioral task for testing the effects of a drug.

At operation 502, a first neuronal cell culture is trained to perform a goal-oriented behavioral task without a drug. The first neuronal cell culture may include any of the types of cells discussed in this disclosure. It may also be in the of the types of cell cultures described above. The goal-oriented behavioral task may be any of the types of tasks discussed in this disclosure.

At operation 504, a second neuronal cell culture is trained to perform the goal-oriented behavioral task with the drug. The second neuronal cell culture includes neurons from a same cell line as the first neuronal cell culture. Thus, the same cells are used to create both cell cultures. For example, this may be the same line of embryonic brain cells or induced pluripotent stem cells created from the same individual. The training is also the same for the second neuronal cell culture. Thus, two different neuronal cell cultures are created with the same neurons and provided the same training. The only functional difference is that the second neuronal cell culture are exposed to the drug during training. However, this technique is not limited to the creation of only two neuronal cell cultures; a larger number of neuronal cell cultures may be created from the same cell line.

At operation 506, the learning abilities of the first neuronal cell culture and the second neuronal cell culture are compared. This compares the ability of the first neuronal cell culture to learn the goal-oriented behavioral task in the absence of the drug to the ability of the second neuronal cell culture to learn the goal-oriented behavioral task in the presence of the drug. Learning abilities that are compared may include the time or number of training cycles needed to learn the goal-oriented behavioral task. This may be thought of as a measure of how quickly or readily the neuronal cell culture is able to learn the task. The comparing may additionally or alternatively compare competency in performing the goal-oriented behavioral task at the completion of training. This is a measure of how well the trained neuronal cell cultures can perform the task.

If additional testing is performed beyond that of comparing a first neuronal cell culture to a second neuronal cell culture, method 500 may proceed to additional testing. Additional testing uses other neuronal cell cultures besides the first neuronal cell culture and the second neuronal cell culture.

One type of additional testing is testing to determine the effects of dose. In such implementations, method 500 proceeds to operation 508 at which point a third neuronal cell culture is trained to perform the goal-oriented behavioral task in the presence of the drug at a different dosage. Thus, the third neuronal cell culture and the second neuronal cell culture both receive the same drug but in different amounts. The third neuronal cell culture includes neurons from the same cell line as the first neuronal cell culture and the second neuronal cell culture. Additional cell cultures (e.g., fourth, fifth, etc.) may also be created to test other dosages.

At operation 510, a dosage of the drug that maximizes the ability of the neuronal cell culture to learn the goal-oriented behavioral task is determined. This may be done simply by comparing the learning abilities of the second neuronal cell culture and the third neuronal cell culture. The dosage applied to the neuronal cell culture which has the greatest learning ability is then identified as the best dosage for maximizing learning abilities. If a larger number of cell cultures testing additional different dosages are used, then the dosage that maximizes learning can be identified from among the tested dosages.

Another type of additional testing is to test the effect of the drug on a variety of different goal-oriented behavioral tasks. For example, a first task may be playing a game in a computer simulation while a second task is solving a machine learning control problem. In such implementations, method 500 would proceed to operation 512 at which point a third cell culture is trained, without the drug, to perform a second goal-oriented behavioral task that is different than the goal-oriented behavioral task used for training at operations 502 and 504.

At operation 514, a fourth neuronal cell culture is also trained to perform the second goal-oriented behavioral task. The fourth neuronal cell culture is trained with the drug. Thus, the third neuronal cell culture and the fourth neuronal cell culture form a pair of otherwise identical cell cultures, one trained with the drug and one without. The neurons used to form the third neuronal cell culture and the fourth neuronal cell culture include cells from a same cell line as the first neuronal cell culture and also the second neuronal cell culture. Thus, the difference between the first neuronal cell culture and the second neuronal cell culture with respect to the third neuronal cell culture in the fourth neuronal cell culture is the training. All four cell cultures are created with the same type of neurons. The first two are trained to perform a first task while the second two are trained to perform a second, different task.

At operation 516, the learning abilities of the third neuronal cell culture and the fourth neuronal cell culture are compared. This compares the ability of the third neuronal cell culture to learn the goal-oriented behavioral task in the absence of the drug to the ability of the fourth neuronal cell culture to learn the goal-oriented behavioral task in the presence of the drug. Learning abilities that are compared may include the time or number of training cycles needed to learn the goal-oriented behavioral task. This may be thought of as a measure of how quickly or readily a neuronal cell culture is able to learn the task. The comparing may additionally or alternatively compare competency in performing the goal-oriented behavioral task at the completion of training. This is a measure of how well a trained neuronal cell culture can perform the task.

Using multiple trained pairs of neuronal cell cultures provides a battery of different tests to obtain a more comprehensive picture of how a drug might affect actual patients. Testing the effect of a drug on cell cultures trained to perform different tasks provides insights into the effects of the drug that is not specific to any single task. Baselines for “normal” behaviors can be established from the performance of the neuronal cell cultures trained in the absence of the drug. Then for each of the different tasks included in the battery tests, deviations from the baseline due to the drug can be identified.

The battery of tests can be expanded to include different types of cells or cell cultures. For example, the effects of a drug may be tested on human neurons as well as mouse neurons. Neuronal cell cultures can also be tested to see how long they retain the ability to perform a learned skill. Another test that can be performed is to see how fast neuronal cell culture can respond to a change point and restore previous abilities. Each type of test in a battery of tests can be evaluated for the time required by the neuronal cell culture to learn in the presence or absence of a drug. The competency and/or learning ability of the neuronal cell cultures in the presence of the drug are compared to a baseline established by neuronal cell cultures trained in the absence of the drug. The metrics that can be evaluated include competency on the task, rate of learning a new task, speed of response to stimuli, and speed to adapt to a new challenge or a new type of request for response.

### Computing Devices and Systems

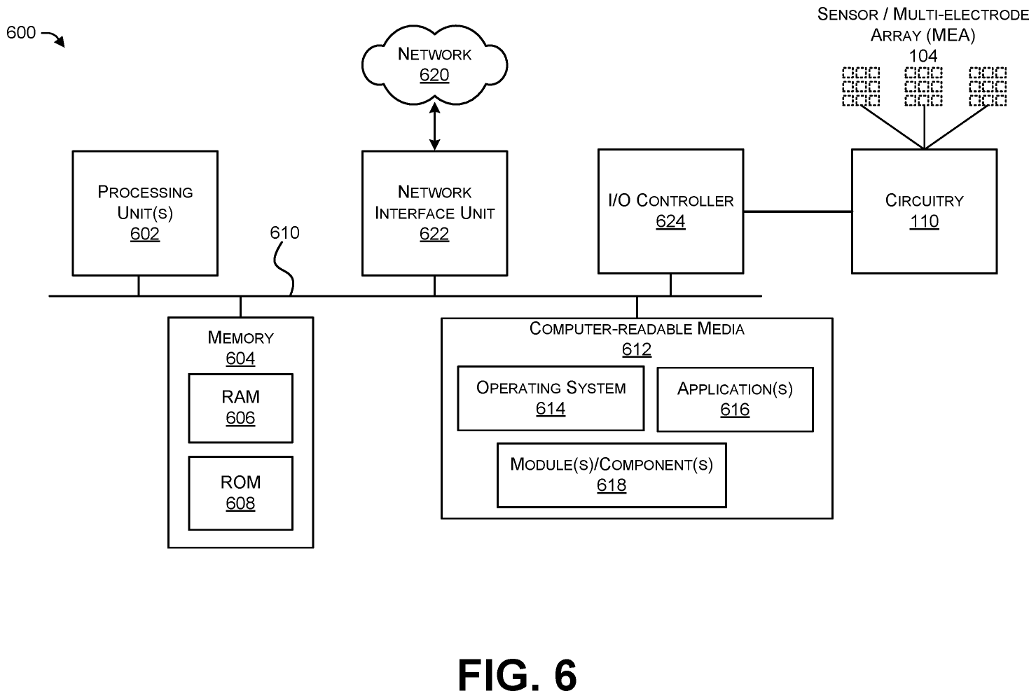

FIG. 6 shows details of an example computer architecture 600 for a computing device such as the computing device 112 introduced in FIG. 1. The computer architecture 600 illustrated in FIG. 6 includes processing unit(s) 602, a memory 604, including a random-access memory 606 (“RAM”) and a read-only memory (“ROM”) 608, and a system bus 610 that couples the memory 604 to the processing unit(s) 602. The processing unit(s) 602 include one or more hardware processors and may also comprise or be part of a processing system. In various examples, the processing units 602 of the processing system are distributed. Stated another way, one processing unit 602 may be located in a first location (e.g., a rack within a datacenter) while another processing unit 602 of the processing system is located in a second location separate from the first location.

The processing unit(s) 602 can represent, for example, a CPU-type processing unit, a GPU-type processing unit, a field-programmable gate array (FPGA), another class of digital signal processor (DSP), or other hardware logic components that may, in some instances, be driven by a CPU. For example, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), and the like.

A basic input/output system containing the basic routines that help to transfer information between elements within the computer architecture 600, such as during startup, is stored in the ROM 608. The computer architecture 600 further includes a computer-readable media 612 for storing an operating system 614, application(s) 616, modules/components 618, and other data described herein. The application(s) 616 and the module(s)/component(s) 618 may implement techniques of this disclosure such as generation of a virtual environment and interpretation of interactions with that virtual environment.

The computer-readable media 612 is connected to processing unit(s) 602 through a storage controller connected to the bus 610. The computer-readable media 612 provides non-volatile storage for the computer architecture 600. The computer-readable media 612 may be implemented as a mass storage device, yet it should be appreciated by those skilled in the art that computer-readable media can be any available computer-readable storage medium or communications medium that can be accessed by the computer architecture 600.

Computer-readable media includes computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including RAM, static random-access memory (SRAM), dynamic random-access memory (DRAM), phase-change memory (PCM), ROM, erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network-attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer-readable storage media does not include communication media. That is, computer-readable storage media does not include communications media and thus excludes media consisting solely of a modulated data signal, a carrier wave, or a propagated signal, per se.

According to various configurations, the computer architecture 600 may operate in a networked environment using logical connections to remote computers through the network 620. The network 620 may be the same as the network 116 shown in FIG. 1. The computer architecture 600 may connect to the network 620 through a network interface unit 622 connected to the bus 610. An I/O controller 624 may also be connected to the bus 610 to control communication in input and output devices. The computing architecture 600 may be coupled through the I/O controller 624 to circuitry 110 which is in turn coupled to sensors and/or stimulators which interact with a neuronal cell culture. In one implementation, the stimulators and sensors may be implemented as a MEA 104 such as that illustrated in FIG. 1.

The MEA 104 may have only a single well or it may be an MEA plate having multiple wells such as 8, 16, 24, 32, 48, or another number of wells. In one implementation, each well in the plate contains 16, 24, 32, 48, or 64 low-impedance (0.04 MU) platinum microelectrodes with 30 mm of diameter spaced by 200 mm. One source of MEA plates is Axion Biosystems of Atlanta, Georgia, USA. A single well may contain one, two, or three, or more organoids. In one implementation, the MEA may be a MaxOne MEA from Maxwell Biosystems, Switzerland that has 26,000 platinum electrodes arranged over an 8 mm² area. The MaxOne system is based on complementary meta-oxide-semiconductor (CMOS) technology and allows recording from up to 1024 channels.

The circuitry 110 may include a bandpass filter and adaptive threshold spike detector. For example, the bandpass filter may be set to 10-25,000 Hz. In another implementation, the bandpass filter may be set to 0.1 Hz to 5 kHz. The adaptive threshold spike detector may be set to 5.5× standard deviations. In one implementation, the circuitry 110 may include a 2nd order high-pass Bessel filter with 100 Hz cut-off followed by a 1st order low-pass Bessel filter with 1 Hz cut-off. Raw data after being passed through the circuitry 110 can be acquired and processed with software such as the Maestro recording system and Axion Integrated Studio available from Axion Biosystems. Alternative software that may be used includes the AxIS Software Spontaneous Neural Configuration also from Axion Biosystems.

It should be appreciated that the software components described herein may, when loaded into the processing unit(s) 602 and executed, transform the processing unit(s) 602 and the overall computer architecture 600 from a general-purpose computing system into a special-purpose computing system customized to facilitate the functionality presented herein. The processing unit(s) 602 may be constructed from any number of transistors or other discrete circuit elements, which may individually or collectively assume any number of states. More specifically, the processing unit(s) 602 may operate as a finite-state machine, in response to executable instructions contained within the software modules disclosed herein. These computer-executable instructions may transform the processing unit(s) 602 by specifying how the processing unit(s) 602 transitions between states, thereby transforming the transistors or other discrete hardware elements constituting the processing unit(s) 602.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A drug testing system comprising: a neuronal cell culture (100), wherein the neuronal cell culture was previously trained to perform a goal-oriented behavioral task; a sensor (104) configured to detect spiking activity in the neuronal cell culture; a stimulator configured to apply stimulation to the neuronal cell culture; a drug delivery mechanism (106) configured to apply a drug (108) to the neuronal cell culture; and circuitry (110) configured to communicatively connect the sensor and stimulator to a computing device. This is an example of a system covering a cell culture that is already trained.

Clause 2. The drug testing system of clause 1, wherein the neuronal cell culture comprises human induced pluripotent stem cells (hiPSCs) or non-human stem cells that are either embryonic stem cells or induced pluripotent stem cells.

Clause 3. The drug testing system of clause 1 or 2, wherein the neuronal cell culture is a two-dimensional (2D) cell culture or a three-dimensional (3D) cell culture.

Clause 4. The drug testing system of clause 1, wherein the neuronal cell culture is an organoid.

Clause 5. The drug testing system of any of clauses 1-4, wherein the sensor and the stimulator are a multi-electrode array (MEA).

Clause 6. The drug testing system of any of clauses 1-5, further comprising the computing device, wherein the computing device is configured to compare spiking activity before and after application of the drug and determine how performance of the neuronal cell culture on the goal-oriented behavioral task changes in response to the drug. This is an example of before and after testing.

Clause 7. The drug testing system of any of clauses 1-5, further comprising: a second neuronal cell culture (302) from a same cell line used for the neuronal cell culture, the second neuronal cell culture trained to perform the same goal-oriented behavioral task; a second sensor configured to detect spiking activity in the second neuronal cell culture; a second stimulator configured to apply stimulation to the second neuronal cell culture; second circuitry configured to communicatively connect the second sensor and second stimulator to the computing device; and the computing device, wherein the computing device is configured to compare spiking activity of the neuronal cell culture in the presence of the drug to spiking activity of the second neuronal cell culture in the absence of the drug and determine how performance of the neuronal cell culture compares to performance of the second neuronal cell culture on the goal-oriented behavioral task. This is an example of a side-by-side comparison with and without a drug.

Clause 8. The drug testing system of any of clauses 1-7, further comprising a cloud-based datastore (118) configured to store data (120) comprising an identifier of the drug, an identifier of a source of the neurons in the neuronal cell culture, and a result of the testing.

Clause 9. A method of drug testing comprising: training a neuronal cell culture to perform a goal-oriented behavioral task; detecting spiking activity of the neuronal cell culture while performing the goal-oriented behavioral task in the absence of a drug; contacting the neuronal cell culture with the drug; detecting spiking activity of neuronal cell culture while performing the goal-oriented behavioral task during or after contact with the drug; and evaluating a change in the ability of the neuronal cell culture to perform the goal-oriented behavioral task in response to the drug. This is an example of a method for testing drugs using one pre-trained cell culture.

Clause 10. The method of clause 9, wherein the neuronal cell culture comprises human induced pluripotent stem cells (hiPSCs) or non-human stem cells in a two-dimensional (2D) cell culture or a three-dimensional (3D) cell culture.

Clause 11. The method of clause 9, wherein the neuronal cell culture is an organoid.

Clause 12. The method of any of clauses 9-11, wherein the goal-oriented behavioral task is a task in a computer simulation of a virtual environment (114).

Clause 13. The method of any of clauses 9-12, wherein the training (200) comprises reinforcement learning (RL) using electrical stimulation of the neuronal cell culture or application of a chemical to the neuronal cell culture as feedback (202).

Clause 14. The method of any of clauses 9-13, wherein the detecting spiking activity of the neuronal cell culture is performed by a multi-electrode array (MEA).

Clause 15. The method of any of clauses 9-14, wherein the neuronal cell culture comprises hiPSCs of a specific patient and the method further comprises: identifying patient-specific effects of the drug by comparing testing results for the hiPSCs with testing results from at least one other neuronal cell culture that does not include cells of the specific patient.

Clause 16. The method of clause 15, further comprising storing a record of the patient-specific effects (118) in a cloud-based datastore (114).

Clause 17. A method of drug testing comprising: training a first neuronal cell culture to perform a goal-oriented behavioral task in the absence of a drug (108); training a second neuronal cell culture to perform the goal-oriented behavioral task in the presence of the drug, wherein the second neuronal cell culture includes neurons from a same cell line as the first neuronal cell culture; and comparing an ability of the first neuronal cell culture to learn the goal-oriented behavioral task in the absence of the drug to an ability of the second neuronal cell culture to learn the goal-oriented behavioral task in the presence of the drug. This is an example of a method for testing drugs based on how the drug affects training of cell cultures.

Clause 18. The method of clause 17, wherein the neuronal cell culture comprises human induced pluripotent stem cells (hiPSCs) or non-human stem cells in a two-dimensional (2D) cell culture or a three-dimensional (3D) cell culture.

Clause 19. The method of clause 17, wherein the neuronal cell culture is an organoid.

Clause 20. The method of any of clauses 17-19, wherein the goal-oriented behavioral task is a task in a computer simulation of a virtual environment (114).

Clause 21. The method of any of clauses 17-20, wherein the training (200) comprises reinforcement learning (RL) using electrical stimulation of the neuronal cell culture or application of a chemical to the neuronal cell culture as feedback (202).

Clause 22. The method of any of clauses 17-21, wherein the comparing compares time or number of training cycles needed to learn the goal-oriented behavioral task. This is an example of testing how long it takes to train.

Clause 23. The method of any of clauses 17-21, wherein the comparing compares competency in performing the goal-oriented behavioral task at completion of training. This is an example of testing how well the task is performed after training.

Clause 24. The method of any of clauses 17-23, further comprising: training a third neuronal cell culture to perform the goal-oriented behavioral task in the presence of the drug at a different dosage, wherein the third neuronal cell culture includes neurons from a same cell line as the first neuronal cell culture (508); and determining a dosage of the drug that maximizes the ability of a neuronal cell culture to learn the goal-oriented behavioral task (510). This is an example of dosage testing.

Clause 25. The method of any of clauses 17-23, further comprising: training a third neuronal cell culture to perform a second goal-oriented behavioral task in the absence of the drug (512); training a fourth neuronal cell culture to perform the second goal-oriented behavioral task in the presence of the drug (514); and comparing an ability of the third neuronal cell culture to learn the second goal-oriented behavioral task in the absence of the drug to an ability of the fourth neuronal cell culture to learn the second goal-oriented behavioral task in the presence of the drug (516). This is an example of a battery of tests.

### CONCLUSION

While certain example embodiments have been described, including the best mode known to the inventors for carrying out the invention, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein. Skilled artisans will know how to employ such variations as appropriate, and the embodiments disclosed herein may be practiced otherwise than specifically described. The accompanying claims and their equivalents are intended to cover such forms or modifications as would fall within the scope and spirit of certain of the inventions disclosed herein.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element (e.g., two different sensors).

In closing, although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

Furthermore, references have been made to publications, patents and/or patent applications throughout this specification. Each of the cited references is individually incorporated herein by reference for its particular cited teachings as well as for all that it discloses.

1. A drug testing system comprising:
a neuronal cell culture, wherein the neuronal cell culture was previously trained to perform a goal-oriented behavioral task;
a sensor configured to detect spiking activity in the neuronal cell culture;
a stimulator configured to apply stimulation to the neuronal cell culture;
a drug delivery mechanism configured to apply a drug to the neuronal cell culture; and
circuitry configured to communicatively connect the sensor and stimulator to a computing device.

2. The drug testing system of claim 1, wherein the neuronal cell culture comprises human induced pluripotent stem cells (hiPSCs) or non-human stem cells that are either embryonic stem cells or induced pluripotent stem cells.

3. The drug testing system of claim 1, wherein the neuronal cell culture is a two-dimensional cell culture or a three-dimensional cell culture.

4. The drug testing system of claim 1, wherein the neuronal cell culture is an organoid.

5. The drug testing system of claim 1, wherein the sensor and the stimulator are a multi-electrode array (MEA).

6. The drug testing system of claim 1, further comprising the computing device, wherein the computing device is configured to compare spiking activity before and after application of the drug and determine how performance of the neuronal cell culture on the goal-oriented behavioral task changes in response to the drug.

7. The drug testing system of claim 1, further comprising:
a second neuronal cell culture from a same cell line used for the neuronal cell culture, the second neuronal cell culture trained to perform the same goal-oriented behavioral task;
a second sensor configured to detect spiking activity in the second neuronal cell culture;
a second stimulator configured to apply stimulation to the second neuronal cell culture;
second circuitry configured to communicatively connect the second sensor and second stimulator to the computing device; and
the computing device, wherein the computing device is configured to compare spiking activity of the neuronal cell culture in the presence of the drug to spiking activity of the second neuronal cell culture in the absence of the drug and determine how performance of the neuronal cell culture compares to performance of the second neuronal cell culture on the goal-oriented behavioral task.

8. The drug testing system of claim 1, further comprising a cloud-based datastore configured to store data comprising an identifier of the drug, an identifier of a source of the neurons in the neuronal cell culture, and a result of the testing.

9. A method of drug testing comprising:
training a neuronal cell culture to perform a goal-oriented behavioral task;
detecting spiking activity of the neuronal cell culture while performing the goal-oriented behavioral task in the absence of a drug;
contacting the neuronal cell culture with the drug;
detecting spiking activity of neuronal cell culture while performing the goal-oriented behavioral task during or after contact with the drug; and
evaluating a change in the ability of the neuronal cell culture to perform the goal-oriented behavioral task in response to the drug.

10. The method of claim 9, wherein the neuronal cell culture comprises human induced pluripotent stem cells (hiPSCs) or non-human stem cells in a two-dimensional cell culture or a three-dimensional cell culture.

11. The method of claim 9, wherein the goal-oriented behavioral task is a task in a computer simulation of a virtual environment.

12. The method of claim 9, wherein the training comprises reinforcement learning (RL) using electrical stimulation of the neuronal cell culture or application of a chemical to the neuronal cell culture as feedback.

13. The method of claim 9, wherein the detecting spiking activity of the neuronal cell culture is performed by a multi-electrode array (MEA).

14. The method of claim 9, wherein the neuronal cell culture comprises hiPSCs of a specific patient and the method further comprises:
identifying patient-specific effects of the drug by comparing testing results for the hiPSCs with testing results from at least one other neuronal cell culture that does not include cells of the specific patient.

15. The method of claim 14, further comprising storing a record of the patient-specific effects in a cloud-based datastore.

16. A method of drug testing comprising:
training a first neuronal cell culture to perform a goal-oriented behavioral task in the absence of a drug;
training a second neuronal cell culture to perform the goal-oriented behavioral task in the presence of the drug, wherein the second neuronal cell culture includes neurons from a same cell line as the first neuronal cell culture; and
comparing an ability of the first neuronal cell culture to learn the goal-oriented behavioral task in the absence of the drug to an ability of the second neuronal cell culture to learn the goal-oriented behavioral task in the presence of the drug.

17. The method of claim 16, wherein the comparing compares time or number of training cycles needed to learn the goal-oriented behavioral task.

18. The method of claim 16, wherein the comparing compares competency in performing the goal-oriented behavioral task at completion of training.

19. The method of claim 16, further comprising:
training a third neuronal cell culture to perform the goal-oriented behavioral task in the presence of the drug at a different dosage, wherein the third neuronal cell culture includes neurons from a same cell line as the first neuronal cell culture; and
determining a dosage of the drug that maximizes the ability of a neuronal cell culture to learn the goal-oriented behavioral task.

20. The method of claim 16, further comprising:
training a third neuronal cell culture to perform a second goal-oriented behavioral task in the absence of the drug;
training a fourth neuronal cell culture to perform the second goal-oriented behavioral task in the presence of the drug; and
comparing an ability of the third neuronal cell culture to learn the second goal-oriented behavioral task in the absence of the drug to an ability of the fourth neuronal cell culture to learn the second goal-oriented behavioral task in the presence of the drug.
